(19) United States
(12) Patent Application Publication
(10) Pub. No.: US 2018/0353722 A1
(43) Pub. Date: Dec. 13, 2018

(54) Aromatherapy Breathing Apparatus

(71) Applicant: Richard Stineman, Coral, PA (US)

(72) Inventor: Richard Stineman, Coral, PA (US)

(21) Appl. No.: 15/618,667

(22) Filed: Jun. 9, 2017

(51) Int. Cl. A61M 16/08; A61M 16/06; A61M 16/14

(52) U.S. Cl. CPC A61M 16/0816; A61M 16/06; A61M 2205/123; A61M 16/14; A61M 16/0875

(57) ABSTRACT

A scent delivery system is provided that may provide for a hollow cylinder having a scent cartridge wherein the present invention may provide that air from a breathing machine's air supply hose pass through the scent cartridge and into a breathing mask. In some embodiments, the scent cartridge be connected inline to a breathing machine in-between the machine's air supply hose and the breathing mask. In some embodiments, the scent cartridge may be located on the outside of the cylinder.

### CROSS-REFERENCE TO RELATED APPLICATION

Not applicable.

### TECHNICAL FIELD

The present invention relates generally to the field of scented therapies and more specifically relates to a scent delivery system for use with a breathing machine.

### BACKGROUND

Many people require assistance from breathing machines such as a continuous positive airway pressure therapy (CPAP) machine or a bilevel positive airway pressure (BIPAP) machine. Such machines persons with obstructive sleep apnea (OSA) breathe more easily during sleep. A CPAP machine increases air pressure in a user's throat so that their airway does not collapse when the user breathes in. A BIPAP machine functions much the same way, but instead uses two levels of air pressure at different times throughout the night. Such machines are intended to relax and assist users in their sleeping habits, but the air in such machines, at best, is no better than the bedroom air, and at worst can smell like metal, plastic, lubricant, or other machine elements. Although some home remedies or inadequate solutions exist, these involve putting drops of scent on a filter in the back of a breathing machine, which eventually will clog it. Therefore, a need exists to treat the air in a CPAP or BIPAP machine (collectively “breathing machines”) with pleasant scents.

Various attempts to solve this problem have been made. One such attempt can be seen with respect to U.S. Patent Application Publication No. 2002/0144684, incorporated by reference in its entirety herein, which generally discloses a mask that can be converted between CPAP and BIPAP uses. While this disclosure does provide for a mask having advanced utility, it fails to provide for an effective scenting system for breathing machines.

Another attempt can be seen with respect to U.S. Pat. No. 7,575,005 incorporated by reference in its entirety herein, which generally discloses a mask having various sensors. While this disclosure does provide for a breathing machine to detect various changes in the user's state, it fails to adequately provide for an effective scenting system for breathing machines.

Yet another attempt can be seen with respect to U.S. Pat. No. 6,842,218, incorporated by reference in its entirety herein, which generally discloses a scent delivery system that pumps a scent through a breathing mask. While this disclosure does provide for the user to receive scented air, it fails to be adequately configured for use with a breathing machine.

Various attempts have been made to solve the problems which may be found in the related art but have thus far been unsuccessful. A need exists for a new scent delivery system for use with a breathing machine to avoid the above-mentioned problems.

### SUMMARY OF THE INVENTION

It is to be understood that in the present disclosure, all embodiments are provided as illustrative and non-limiting representatives of many possible embodiments. In addition, the terms “is,” “can,” “will,” and the like are herein used as synonyms for and interchangeable with terms such as “may,” “may provide for,” and “it is contemplated that the present invention may” and so forth.

The present invention relates to a scent delivery system for use with a breathing machine that overcomes the deficiencies of the prior art. It is contemplated that the present invention may provide a breathing machine accessory that provides scents to users.

For purposes of summarizing, certain aspects, advantages, and novel features of the present invention are provided herein. It is to be understood that not all such aspects, advantages, or novel features may be provided in any one particular embodiment. Thus, the disclosed subject matter may be embodied or carried out in a manner that achieves or optimizes one aspect, advantage, or novel feature or group of features without achieving all aspects, advantages, or novel features as may be taught or suggested.

In view of the foregoing disadvantages inherent in the known art, the present invention provides a scent delivery system for use with a breathing machine. The general purpose of the present invention, which will be described subsequently in greater detail, is to provide a user with pleasant scents while drifting off to sleep. The features of the invention which are believed to be novel are particularly pointed out and distinctly claimed in the concluding portion of the specification. These and other features, aspects, and advantages of the present invention will become better understood with reference to the following drawings and detailed description.

In some embodiments, the present invention may provide for a hollow cylinder configured to connect with a breathing machine's air supply line on one end of the cylinder connector element, and to a breathing mask on the other end of the cylinder connector element. A cartridge containing a scent may be, in some embodiments, located within the cylinder connector element (an “inline” embodiment). In other embodiments, the cartridge may be on the outside of the cylinder (an “outside” embodiment). In some inline embodiments, an air inlet element may intake air from the air supply hose and transfer that air to the cartridge, which may then pass the air through an air outlet element, whereupon the air may then pass into the breathing mask. In the outside embodiments, an air outlet element may pass the air from inside the cylinder element out to the outside cartridge. The air may then pass through the cartridge and into an air inlet element, that may allow the air to pass back into the cylinder connector element and then into the breathing mask.

In one embodiment, the present invention may provide for a scent delivery system for use with a breathing machine. In some embodiments, the present invention may provide for an inline accessory configured to be secured between a breathing machine's hose and the breathing machine's mask. In some embodiments, the present invention may provide for an airtight seal, or a nearly airtight seal, on both sides of the cartridge so as to maximize the oxygen and other air flow of a breathing machine. The present invention's cartridge may have with male/female ends to fit into air supply line of CPAC/BIPAP machine with a small scent canister that is removed easily. The present invention's scent canister may provide for a variety of scents.

In some embodiments, the present invention may provide for a small cylindrical piece of plastic pipe, which in some embodiments may be approximately three inches long, the pipe piece element (the “canister”) having a male end and a female end. The present invention may provide for one or more scent cartridges that in some embodiments may connect to the top of the pipe piece element, wherein the scent cartridge element may have a hole so the air will flow from the breathing machine's hose through the present invention and into the mask of a user. In some embodiments the present invention may simulate, approximate, or provide for an air conduit or line that may act like a vacuum.

In some embodiments, the present invention's cylindrical pipe piece element may have a male connecting element and a female connecting element. It is contemplated that the present invention may connect between a breathing machine's air supply line and a breathing mask. In some embodiments, the present invention may provide that the present invention's male connecting element may connect with the breathing mask and the female connecting element may connect with the air supply line, or vice versa. In some embodiments, the cartridge may plug into the canister. In other embodiments, the cartridge may twist into or be threadedly connected to the canister. The canister and the cartridge may also be supplemented with additional design elements as well.

The unique features of the present invention may provide the following benefits for one or more consumers: it may take away some of the anxiety of wearing a mask by giving a more soothing environment with a scent of the individual's choice; may make the assisted breathing experience more pleasant; may be easy to install; may provide for different scents; and may be affordable.

### BRIEF DESCRIPTION OF THE DRAWINGS

These and other features, aspects and advantages of the present invention will become better understood with regard to the following description, appended claims, and accompanying figures where:

FIG. 1 shows a top perspective view of a scent delivery system in accordance with one embodiment of the invention.

FIG. 2 shows a top perspective view of a scent delivery system in accordance with one embodiment of the invention.

FIG. 3 shows a close-up perspective view of a scent delivery system in accordance with one embodiment of the invention.

FIG. 4 shows an elevational view of a scent delivery system in accordance with one embodiment of the invention.

FIG. 5 shows a plan view of a scent delivery system in accordance with one embodiment of the invention.

FIG. 6 shows a top perspective view of a scent delivery system in accordance with one embodiment of the invention.

### DETAILED DESCRIPTION

The present invention overcomes the limitations of the prior art by providing a new and more effective scent delivery system.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any embodiment or element of an embodiment disclosed in this disclosure will be determined by its intended use.

It is to be understood that the drawings and the associated descriptions are provided to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific detail. Well known features, elements or techniques may not be shown in detail in order not to obscure the embodiments.

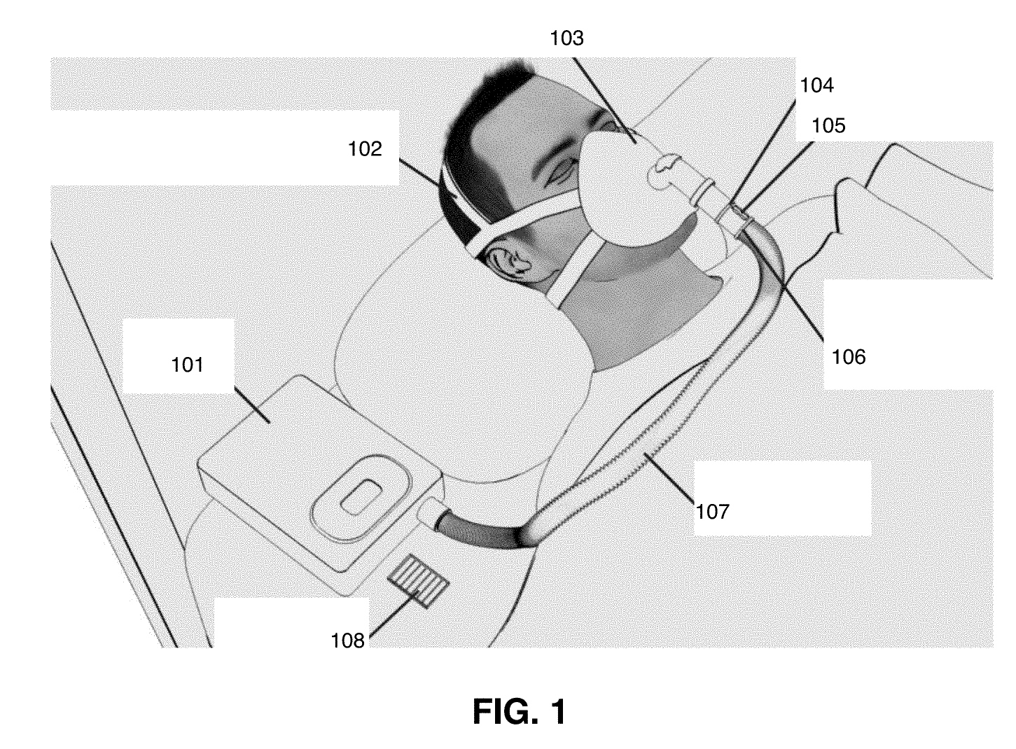

Turning attention to FIG. 1, a top perspective view of a scent delivery system in accordance with one embodiment of the invention is shown. In the embodiment depicted, a viewer may perceive oxygen machine 101, head strap 102, oxygen mask 103, male hose connection 104 (which may be elastomer), scent cartridge 105, female hose connection 106 (which may be elastomer), rubber hose 107, and plastic case 108.

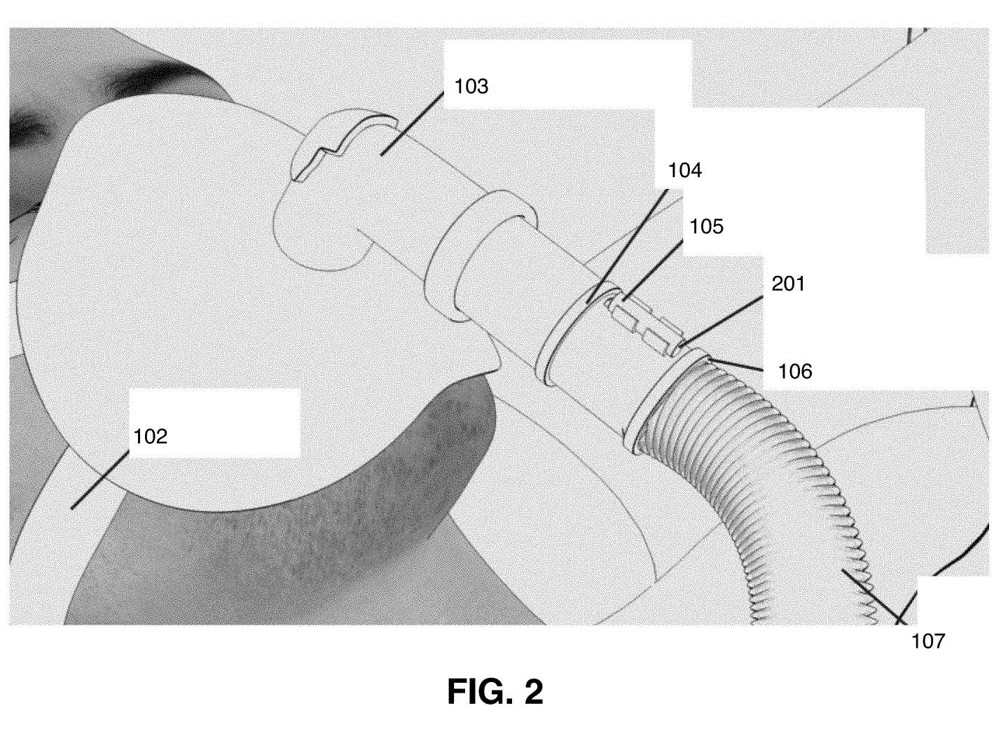

With respect to FIG. 2, a top perspective view of a scent delivery system in accordance with one embodiment of the invention is shown. In the embodiment depicted, a viewer may perceive air inlet 201, along with head strap 102, oxygen mask 103, male hose connection 104 (which may be elastomer), scent cartridge 105, female hose connection 106 (which may be elastomer), and rubber hose 107.

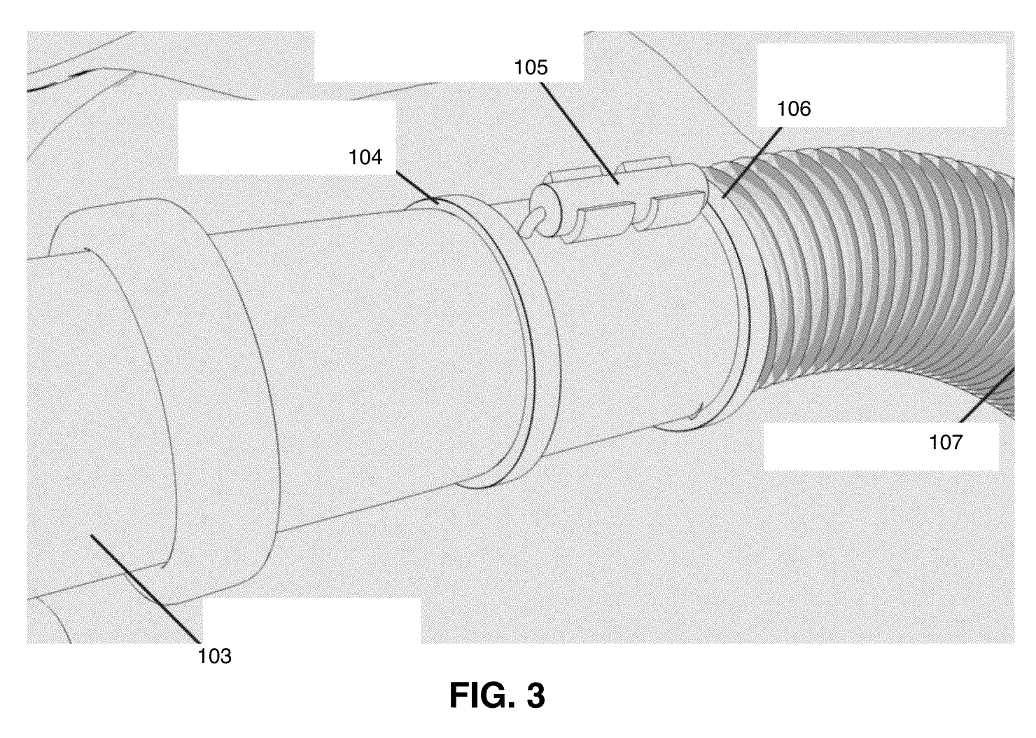

With respect to FIG. 3, a close up perspective view of a scent delivery system in accordance with one embodiment of the invention is shown. In the embodiment depicted, a viewer may perceive oxygen mask 103, male hose connection 104 (which may be elastomer), scent cartridge 105, female hose connection 106 (which may be elastomer), and rubber hose 107.

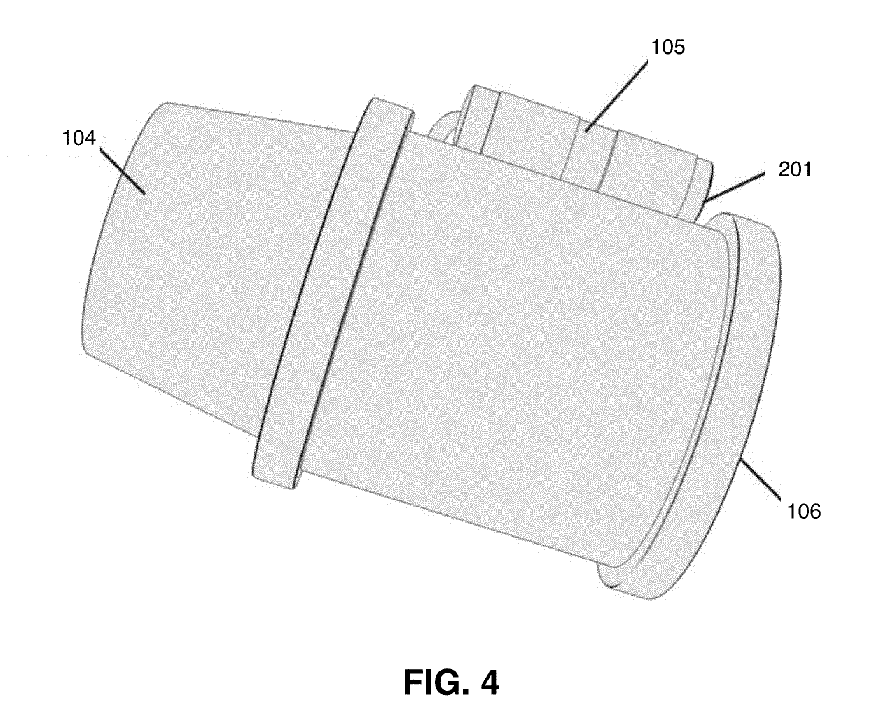

With respect to FIG. 4, an elevational view of a scent delivery system in accordance with one embodiment of the invention is shown. In the embodiment depicted, a viewer may perceive air inlet 201, male hose connection 104 (which may be elastomer), scent cartridge 105, and female hose connection 106 (which may be elastomer).

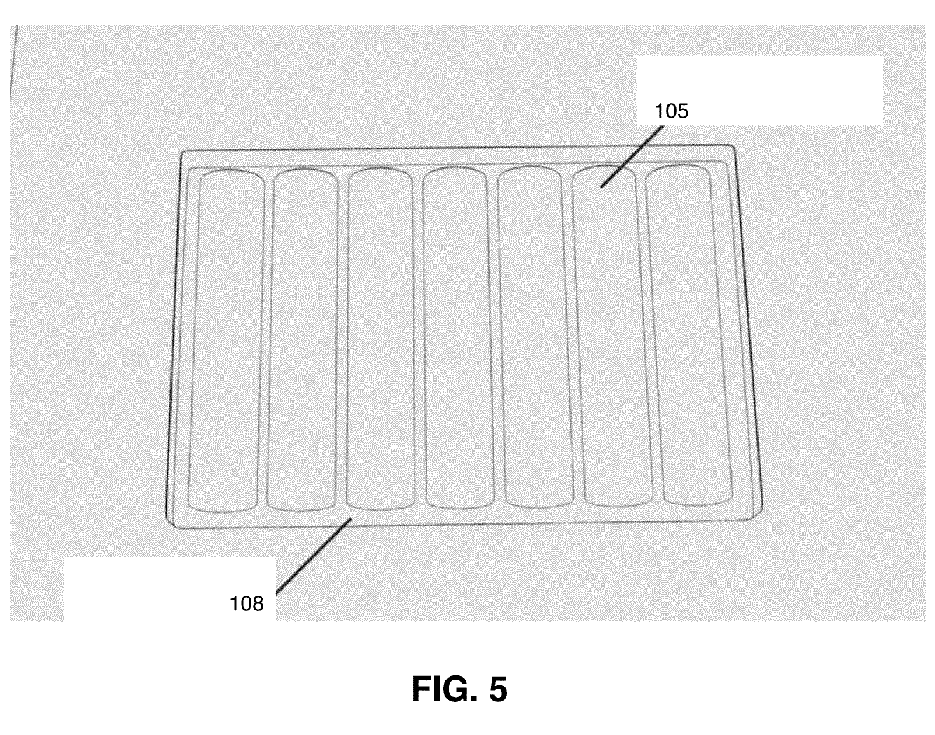

With respect to FIG. 5, a plan view of a scent delivery system in accordance with one embodiment of the invention is shown. In the embodiment depicted, a viewer may perceive scent cartridge 105 and plastic case 108.

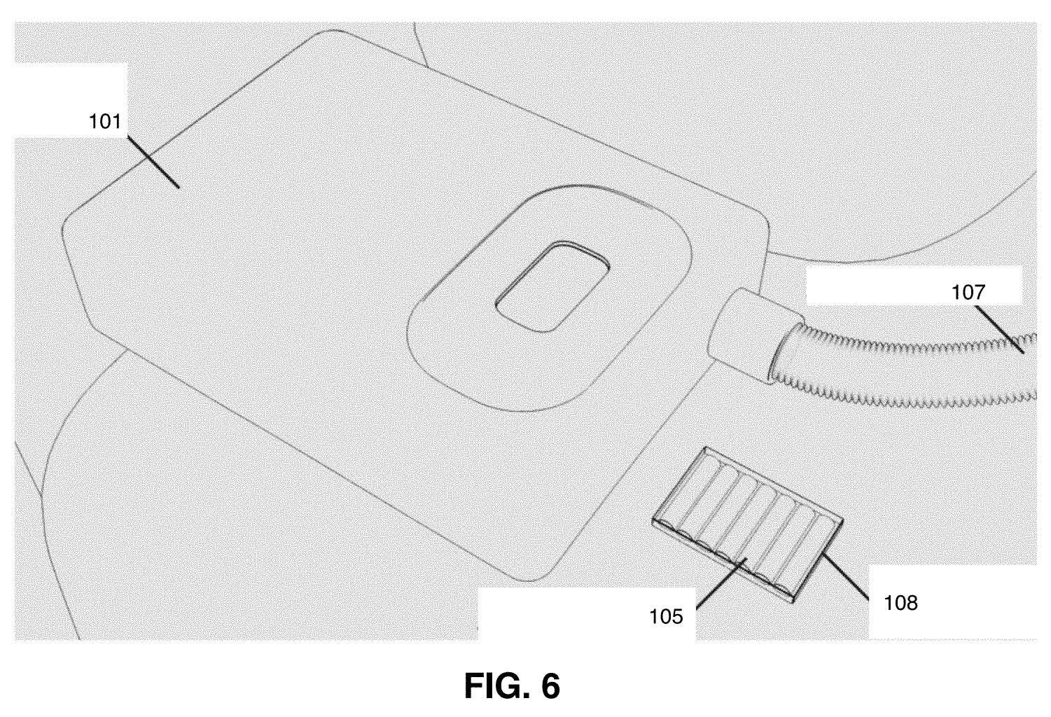

With respect to FIG. 6, a top perspective view of a scent delivery system in accordance with one embodiment of the invention is shown. In the embodiment depicted, a viewer may perceive oxygen machine 101, scent cartridge 105, rubber hose 107, and plastic case 108.

### CONCLUSIONS, RAMIFICATIONS, AND SCOPE

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

While the scent delivery system generally described herein has been disclosed in connection with a number of embodiments shown and described in detail, various modifications should be readily apparent to those of skill in the art.

What is claimed is:

1. A scent delivery system, comprising:
a hollow cylindrical connector element, the connector element having a first end and a second end, wherein the first end is configured to connect to a breathing mask and the second end is configured to connect to an air supply hose;
an air outlet element, wherein the air outlet element is configured to allow air from inside the connector element to pass out of the inside of the connector element;
an air inlet element, wherein the air inlet element is configured to allow air from outside the connector element to pass into the connector element; and
a cartridge, wherein the cartridge is configured to receive air from the air outlet element and pass the air into the air inlet element, and wherein the cartridge is configured to dispense at least one scent.

2. The system of claim 1, further comprising a plastic tray configured to removably retain one or more cartridges.

3. The system of claim 1, wherein the first end is configured to support an airtight seal with a breathing mask and the second end is configured to support an airtight seal with an air supply hose.

4. A scent delivery system, comprising:
a hollow cylindrical connector element, the connector element having a first end and a second end, wherein the first end is configured to connect to a breathing mask and the second end is configured to connect to an air supply hose;
an air inlet element;
an air outlet element; and
a cartridge, wherein the cartridge is configured to receive air from the air inlet element and pass the air into the air outlet element, and wherein the cartridge is configured to dispense at least one scent.

5. The system of claim 4, further comprising a plastic tray configured to removably retain one or more cartridges.

6. The system of claim 4, wherein the first end is configured to support an airtight seal with a breathing mask and the second end is configured to support an airtight seal with an air supply hose.
